(19) United States
(12) Patent Application Publication
(10) Pub. No.: US 2011/0071425 A1
(43) Pub. Date: Mar. 24, 2011

(54) Laser-perforated intra-parenchymal micro-probe

(75) Inventors: Nandor Ludvig, New York, NY (US); Walter Blumenfeld, Airville, PA (US); Geza Medveczky, New York, NY (US)

(73) Assignee: Lenox Laser Corporation, Glen Arm, MD (US)

(21) Appl. No.: 12/586,300

(22) Filed: Sep. 21, 2009

(51) Int. Cl. A61B 10/02; A61M 1/00; A61N 1/36

(52) U.S. Cl. 600/562; 604/35; 604/27; 604/31; 604/21

(57) ABSTRACT

Apparatus and methods in which very small volumes of biological fluid-borne material, particularly large molecules such as proteins, may be selectively extracted from or delivered to interstitial fluid (in vivo or in vitro) by means of intra-parenchymal micro-probes inserted in the brain. The primary use of the micro-probe is in neuroscience research, clinical diagnostics or treatment of epilepsy and other neurological conditions; it may also be applied to other organs and biological systems. Eventual human clinical applications may include neurosurgical monitoring, functional tracking of devices or materials introduced in a surgical procedure, or cerebro-spinal fluid sampling.

### FIELD OF THE INVENTION

{The field of the invention is medical and neuroscience instrumentation for research, clinical diagnostics and therapy, particularly for size-selective molecular sampling and delivery of fluid-borne agents to and from interstitial fluid in the brain, or in cerebro-spinal fluid in the spinal column.}

### BACKGROUND

A micro-probe capable of sampling and delivery of relatively large particles, such as protein molecules, cells and microorganisms with minimal fluid transfer or trauma to selected sites in the brain may be of great utility in neuroscience research, clinical diagnostics or treatment of epilepsy and other neurological conditions.

Relevant prior art includes the use of the push-pull cannula, which comprises two adjacent, open-end cannulae with one cannula carrying the “pushed fluid” downward, whereas the other one carrying the “pulled fluid” upward, creating an open molecule-exchange zone at the tip of the two cannulas. This method was widely used in the sixties and seventies; it then became clear that the technique has the serious problems of frequent clogging of the cannulae by tissue or by clotting of fluids and damage to tissue by fluid build-up around the open-end cannula-tips. Alternative prior art teaches the use of microdialysis probes. The innovation of the microdialysis probe was the replacement of the open-end cannula-tips of the push-pull method with a microdialysis probe or fiber containing a semi-permeable membrane. This eliminated the blockage of perfusion inside the cannulae and prevented unwanted tissue damage. Yet, the very innovation that gave birth to this technique, the use of the microdialysis membrane, led to another problem: the inability to collect and deliver large particles, including such critical biological substances as proteins; these particles and molecules are very large and cannot pass through the membrane. It has become clear that new approaches are needed, and this micro-probe is a response to this need.

### BRIEF SUMMARY OF THE INVENTION

The Laser-Perforated Intra-Parenchymal Micro-Probe (“LAPP”) comprises a fluid manifold body having inlet and outlet ports connected respectively to the interior volume of a nested, coaxial dual-lumen cannula or microtube, in which the inlet port feeds the inner cannula, and the outlet port drains the annular volume (external to the inner cannula and internal to the outer cannula), such that the tip of the outer cannula is sealed, and the only fluid access between fluid inside the microtube assembly and the external biological tissue in which the microtube is inserted is provided by an array of laser-perforated apertures having a uniform size selected to enable extraction of molecules or fluid-borne material, but excluding any material of size greater than that of the apertures. Conversely, the aperture size also allows delivery of size-limited fluid-borne material. Connection of the inlet and outlet ports to independently programmable fluid pumps allows operation of the micro-probe according to a variety of protocols, enabling sampling (extraction) or delivery of fluid-borne material with net zero or non-zero fluid volume extracted or delivered, along with positive sampling or delivery of the fluid-borne molecules or material.

The Laser-Perforated Intra-Parenchymal Micro-Probe provides a minimally invasive means for sampling and delivery of picoliter/microliter fluid volumes, with selective size control on transfer of suspended material or molecules. These micro-probes, herein referred to as the “device”, provide alternatives to and significant improvements on current microdialysis membrane probe technology; these improvements relate to (1) greater dynamic selectivity range for transferred molecule (or particle) size, (2) pressure-augmented diffusion-driven molecular (or particulate) transfer capability, (3) durability under fluid pressure and mechanical force, (4) service lifetime and (5) tolerance of cleaning procedures for repeated use. This invention was developed with the aid of NIH grants #1R43NS049714-01, #9R44 MH080693-02 and #5 R44 MH080693-03.

### BRIEF DESCRIPTION OF THE DRAWINGS

FIG. 1 shows a schematic of a preferred embodiment of the invention.

FIG. 2 details the active sampling and delivery portion of the micro-probe, with an exterior view (A) and an interior schematic (B).

FIG. 3 is a representation of the Mode 2, Mode 3, and Mode4 operating protocol for the use of the invention.

FIG. 4 is a representation of the bolus delivery of a diagnostic or therapeutic agent with a Mode 5 operating protocol.

FIG. 5 is a representation of a (non-volume-replaced) fluid sample extraction with a Mode 5 operating protocol.

FIG. 6 illustrates a standard lumbar puncture procedure (A) and a lumbar puncture procedure utilizing the LAPP invention.

### DETAILED DESCRIPTION OF THE INVENTION

### Intra-Parenchymal Micro-Probe Structure

The device comprises multiple sections of thin-wall tubing, retained in intersecting bores in a multi-port manifold body. The manifold and tubing provide access for fluid extraction or delivery. The manifold body may be fabricated from stainless steel, titanium, ceramic, glass, acetyl, or some other biocompatible material. The tubing must also be a biocompatible material, not necessarily the same as that of the manifold body. Appropriate material selection allows fabrication of probes which are compatible with MRI and other diagnostic procedures. The laser-perforated design has the ability to size-selectively exclude materials from extracted or delivered fluid; it may also minimize tissue damage at the sampling or delivery site by distributing the fluid volume interface over multiple small orifices covering a much larger area than a plain needle tip.

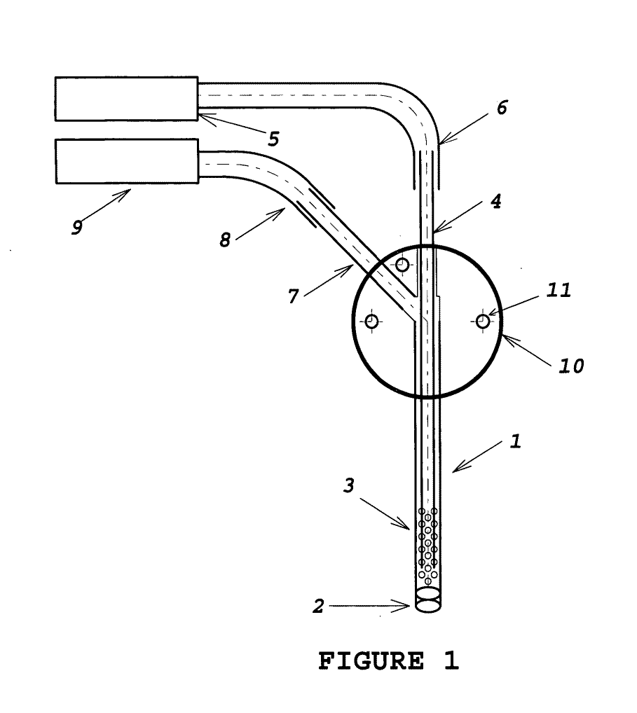

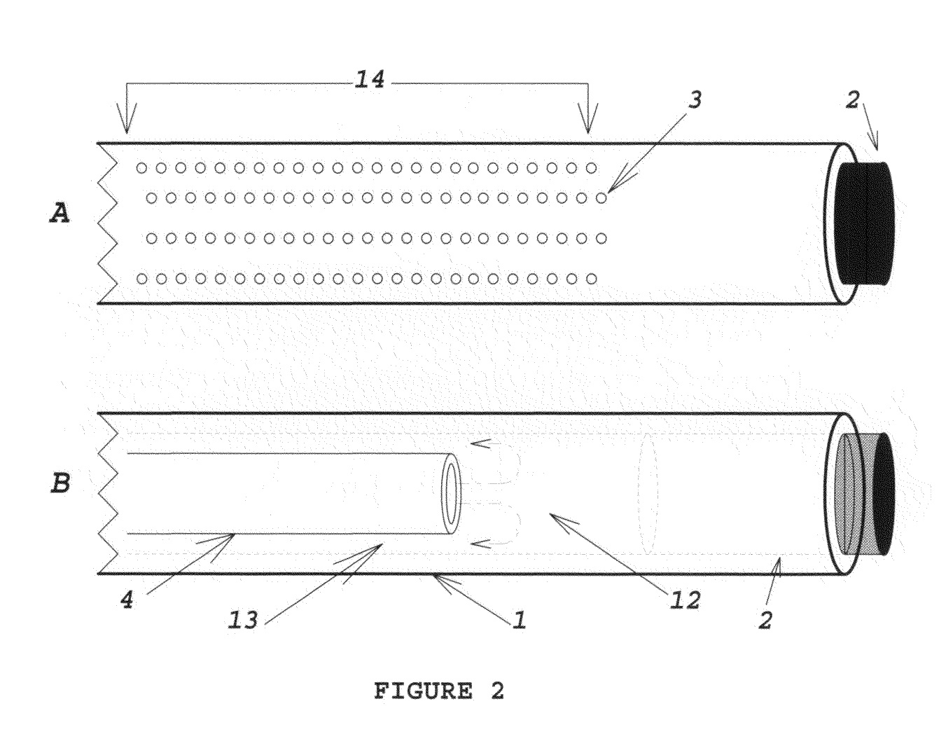

FIG. 1 illustrates a preferred embodiment of the invention. The functional portion of the device is the main microtube 1, a section of stainless steel hypodermic tubing (typically 27-gauge thin-wall having a typical working length from 25 mm to 100 mm) which is inserted into the tissue site of interest. The tip of the main microtube 1 is sealed with a tip plug 2, which is formed from a weld, a short wire or adhesive filler. With reference to FIG. 2, an array 3 of laser-drilled apertures is located in {a selective transfer area 14 on} the cylindrical surface of the main microtube 1 near the tip plug 2; this array 3 may be asymmetric (for example, a single column of holes) or uniformly distributed (as in a regular cylindrical array). Fluid is supplied to the laser perforation array 3 through a (typically 33-gauge) coaxial inlet tube 4 by a software-controlled delivery pump 5 via the delivery port 6. Fluid is extracted from the laser perforation array 3 through the annular-cross-section volume (between the outside of the coaxial inlet tube 4 and the inside of the main microtube 1) via the oblique outlet tube 7 and the sampling port 8 by a software-controlled extraction pump 9. The main microtube 1, coaxial inlet tube 4, and the oblique outlet tube 7 are mounted in the manifold body 10, which is a rigid disk fabricated of biocompatible material such as acetyl provides mechanical stabilization and fluid seals. The assembly may be attached to other instrumentation with the aid of three mounting pin through-holes 11.

### Intra-Parenchymal Micro-Probe Function

In the simplest steady-state operation mode, the delivery pump 5 and sampling pump 9 are driven at identical, non-zero volume-controlled flow rates. Fluid is forced through the delivery port 6, down the coaxial inlet tube 4 and exits into the interior of the main microtube 1 at the flow reversal region 12. It is then drawn through the annular flow region 13 until it flows out through the oblique outlet tube 7 and exits through the sampling port 8 into the sampling pump 9. Fluid in internal contact with the laser-drilled aperture array 3 in the selective transfer area 14 may transfer molecules or suspended material to or from the fluid environment outside of the main microtube 1, provided that said molecules or suspended material are smaller than the size of the laser-drilled apertures. The transfer of molecules or suspended material across the selective transfer area 14 may be driven in several modes by diffusion and/or local differential fluid pressure. These transfer modes are dependent on the operating protocol for the delivery pump 5 and sampling pump 9.

### Operating Modes for Material Transfer

### Mode 1: Zero Flow, Equal Fluid Pressure Inside and Outside.

Diffusion transfer rate proportional to difference in concentration is expected, resulting in exponential time-decay to asymptotic concentration balance.

Mode 2: Constant Non-Zero Identical Delivery and Sample Flow, with Equal Fluid Pressure Inside and Outside the Aperture Array.

Diffusion transfer is expected to be proportional to local difference in concentration, with temporal asymptotic approach to dynamic equilibrium of concentration as a function of linear position in aperture array with respect to local flow axis.

### Mode 3: Identical Pulsed “Mirror-Image” Delivery and Sample Flow.

Diffusion transfer is expected to be proportional to local difference in concentration as in mode 1 and mode 2, modified by a monotonic function of instantaneous flow rate.

Mode 4: Asymmetric Pulsed Delivery and Sample Flow, with Identical Mean Delivery and Mean Sample Flow Rates, but with Phase Differences Between Delivery and Sample Flow Pulses.

In this case, diffusion transfer is augmented by temporary non-zero volume exchange and mixing. This is expected to result in greater molecular transfer than mode 3, but will require a more complex representation or model. This mode involves more risk of tissue damage if the temporary non-zero net volume exchange is allowed to be too large; it also offers the potential advantage of reducing the risk of aperture obstruction by intermittent differential-pressure-driven flow through each orifice.

Mode 5: Unbalanced delivery and sample flow, with non-zero net fluid volume delivery or sampling. This mode includes the obvious degenerate cases of delivery-only and sample-only operation, but also allows modifications of modes 2, 3 and 4, with the addition of a single-dose or regular repetitive bolus of active material for diagnostic or therapeutic purposes. This mode may be particularly useful for effecting closed-loop control based on information derived from a real-time sensor attached to the microprobe or system.

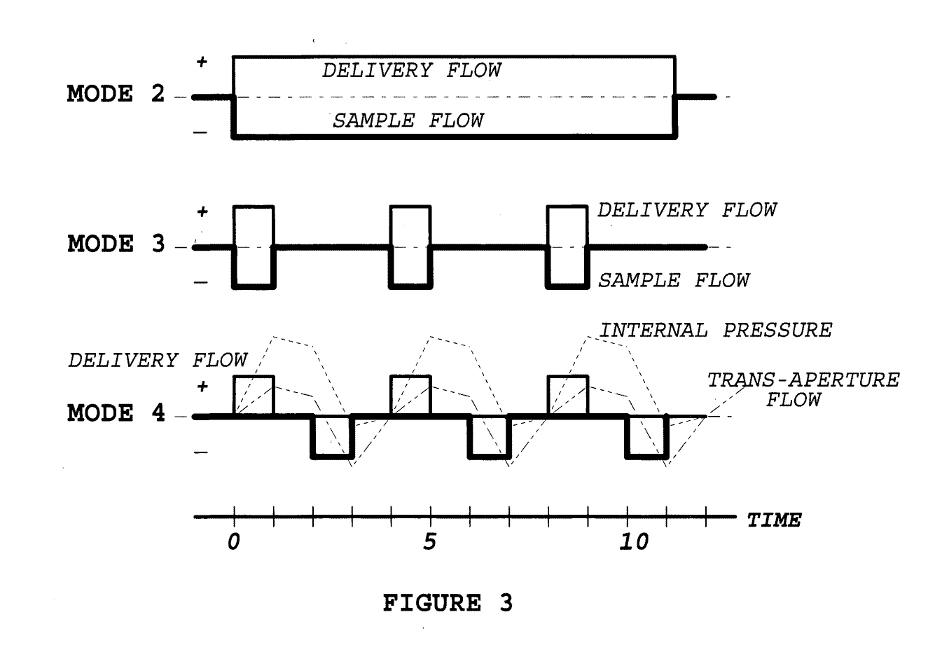

Some examples of pump flow protocols associated with operating modes 2, 3 and 4 are depicted in FIG. 3. The delivery pump flow rate and sample pump flow rate are presented for modes 2, 3 and 4. The mode 4 example also displays representative signals for internal pressure and trans-aperture flow rate.

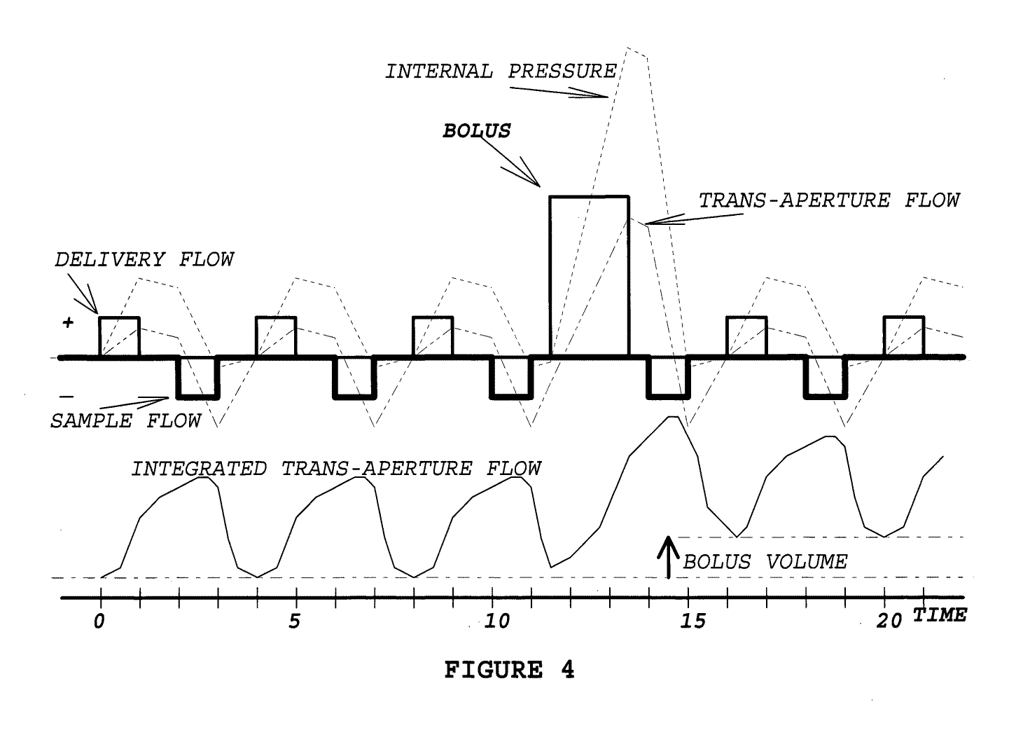

An example of a bolus delivery protocol in operating mode 5 is presented in FIG. 4. Note that the time-integrated trans-aperture flow rate becomes positive after injection of the delivered bolus.

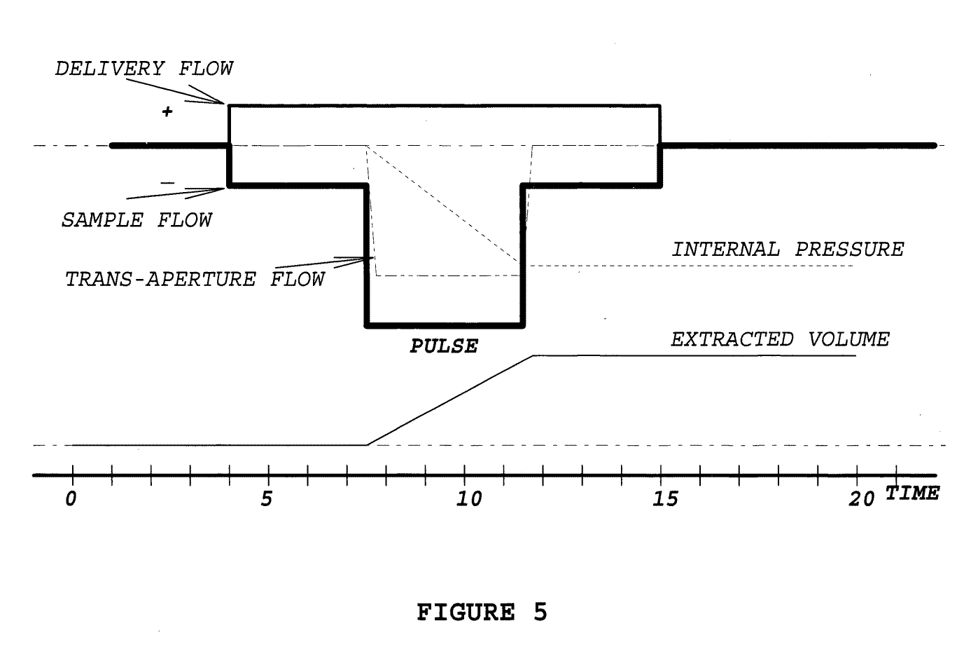

Another example of operating mode 5 is presented in FIG. 5. Here a constant delivery flow rate is coupled with alternating sampling flow rates, where a constant sample flow rate is periodically increased to a higher pulse flow rate, leading to facilitated diffusion of solutes and solvents from the surrounding medium into the lumen of the microprobe.

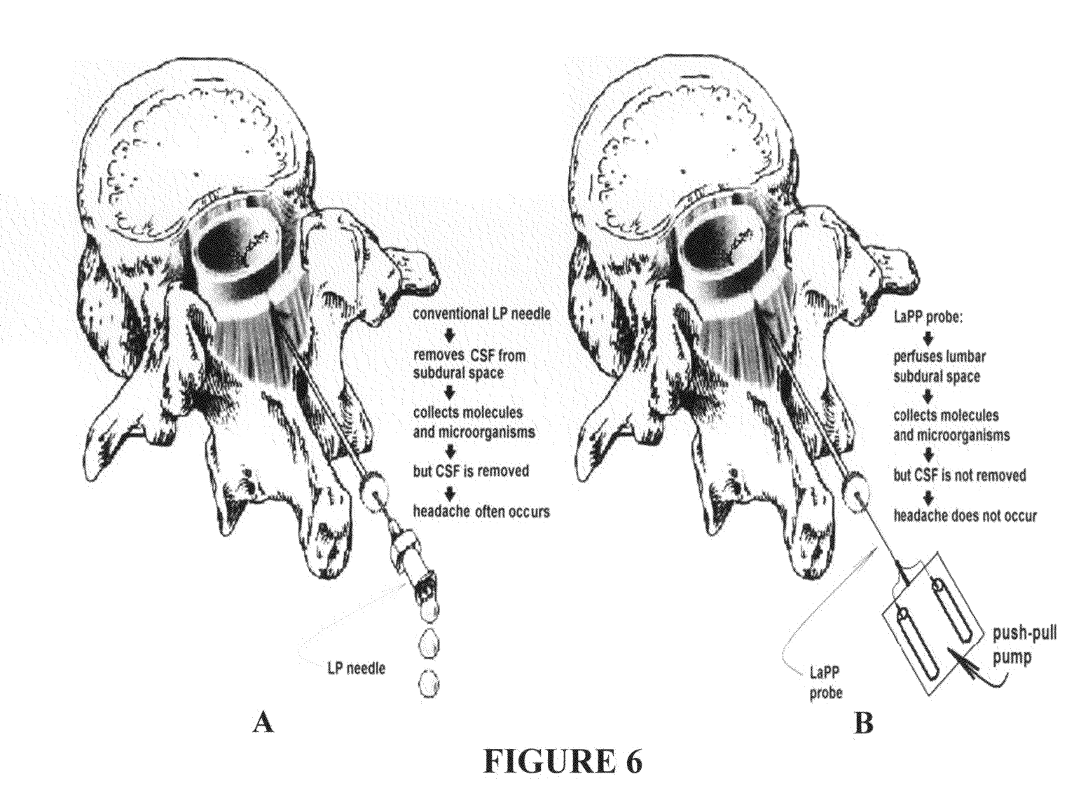

FIG. 6} shows a lumbar puncture application for neurological diagnosis. Current usage (A) removes a considerable volume of cerebrospinal fluid (CSF) for testing. This may cause headache and may be contraindicated in some medical conditions. In contrast, the present invention invention (B) does not need volume removal of CSF for analysis, as it allows the diffusion of large particles, including proteins, cells, bacteria and viruses into carrier fluid in the probe lumen for subsequent analysis.

1. A micro-probe device for use in neuroscience, biotechnology and medical applications which provides access to very small volumes of tissue and/or fluid, primarily for size-selective collection or delivery of fluid-borne material; this material includes large molecules such as proteins, as well as cells and other biological particles. The device comprises a multiport manifold body having at least 2 intersecting bores linking the multiple ports, in which access to the site of interest is combined via the multiport manifold into a single main microtube needle for in-vitro or in-vivo penetration of tissue or fluid to the site of interest. The device provides fluid access (including but not limited to physical extraction and/or delivery) to the site of interest via a selective transfer area. The selective transfer area may be a laser-perforated array of apertures, a porous plug, a screen, a grid, or an array of nanotubes located in the tip or sidewall of the main microtube.

2. The device specified in claim 1, in which physical fluid extraction and for delivery are provided by attached single or multiple micro-perfusion, syringe or peristaltic pumps capable of both supplying and removing fluids to and from the micro-probe.

3. The device specified in claim 1, in which physical fluid extraction and/or delivery is provided by a gravity-feed reservoir system, a vacuum-driven fluidic system or a pneumatic-driven fluidic system.

4. The device specified in claim 1, in which fluid pressure is sampled by a pressure transducer mounted integral to one of the multiple ports on the manifold body or remotely via a tubing connection.

5. The device specified in claim 1, in which the locus of interest is parenchymal tissue in the brain

6. The device specified in claim 5, in which the period of interest includes a seizure or other event of interest.

7. The device specified in claim 6, in which information generated by means of the probe is used to control selection of, volume of, or delivery rate of medication or other biologically active agent.

8. The device specified in claim 2 or claim 3, in which one or more of the pumps or mechanisms required for fluid delivery or sampling is mounted on or integrated with the probe and operated remotely by mechanical, fluidic, pneumatic, electrical or wireless means.

9. The device specified in claim 5, in which the application is delivery of fluid or suspended material as part of a stem cell procedure.

10. The device specified in claim 5, in which the application is delivery of fluid or suspended material as part of a gene therapy procedure.

11. The device specified in claim 1, in which access from within the main microtube to the external site of interest is an array of apertures having specified geometry produced by a process of laser micro-machining, photo-lithography or etching.

12. The device specified in claim 5 in which physical means for measurement and control are included, along with a microcontroller and a wireless or wired communications link.

13. The device specified in claim 5, in which the size of molecules or suspended particles sampled or delivered through the selective transfer area controlled by the size of the apertures in the selective transfer area.

14. The device specified in claim 5, in which a set of two or more such probes are applied simultaneously to an organ, area or volume of tissue, in order to deliver active agents and/or extract samples which cannot be accessed simultaneously by a single microprobe.

15. The device specified in claim 5, in which all materials used for fabrication are compatible with Magnetic Resonance Imaging in human or animal tissue.

16. The device specified in claim 15, in which the main microtube, inlet tube, and outlet tube are fabricated of Titanium tubing, and in which the manifold body is fabricated of Acetyl (Delrin).

17. The device specified in claim 1, in which the main microtube is also used as a contact for electrical signal measurement or stimulation.

18. The device specified in claim 1, in which the microprobe is used for lumbar puncture to collect molecules, cells and/or microorganisms from the cerebrospinal fluid without the need of removing fluid for analysis, thus eliminating potential adverse affects of the lumbar puncture procedure.
